(19) United States
(12) Patent Application Publication
(10) Pub. No.: US 2022/0406206 A1
(43) Pub. Date: Dec. 22, 2022

(54) RECORDING MEDIUM RECORDED WITH CARDIOPULMONARY RESUSCITATION TRAINING PROGRAM, CARDIOPULMONARY RESUSCITATION TRAINING METHOD, APPARATUS, AND SYSTEM

(71) Applicant: Akinori MINAZUKI, Hokkaido (JP)

(72) Inventors: Tomoki KURIYAGAWA, Hokkaido (JP); Akinori MINAZUKI, Hokkaido (JP)

(21) Appl. No.: 17/444,600

(22) Filed: Aug. 6, 2021

(30) Foreign Application Priority Data
Jun. 17, 2021 (JP) 2021-100838

(51) Int. Cl. G09B 5/02; G06K 9/00; A61B 5/107

(52) U.S. Cl. CPC G09B 5/02; G06K 9/00342; G06K 9/00771; A61B 5/1071; A61B 5/1079; G09B 23/288

(57) ABSTRACT

Disclosed is a non-transitory computer-readable recording medium recorded with a cardiopulmonary resuscitation training program executable by a processor of an information processing apparatus, the cardiopulmonary resuscitation training program causing the processor to perform operations including evaluating a posture of a person who is performing chest compressions, based on posture information indicating the posture obtained from a posture detection apparatus and ideal posture information indicating an ideal posture for the chest compressions stored in a storage unit, to yield an evaluation result, and displaying the evaluation result on a display apparatus.

### CROSS-REFERENCE TO RELATED APPLICATION

The present application is based on and claims the priority to Japanese Patent Application No. 2021-100838 filed on Jun. 17, 2021, the entire content of which is incorporated herein by reference.

### BACKGROUND OF THE INVENTION

### 1. Field of the Invention

The present invention relates to a recording medium recorded with a cardiopulmonary resuscitation training program, a cardiopulmonary resuscitation training method, a cardiopulmonary resuscitation training apparatus, and a cardiopulmonary resuscitation training system.

### 2. Description of the Related Art

Conventionally, a support system for appropriately performing chest compressions in cardiopulmonary resuscitation has been known. Specifically, for example, conventionally, there has been known a technique for instructing music playback and warning output depending on whether or not the compression speed and the compression depth in chest compression training are within predetermined appropriate ranges.

### CITATION LIST

### Patent Literature

PTL 1: Japanese Laid-Open Patent Publication No. 2017-211436

### SUMMARY OF THE INVENTION

### Technical Problem

The conventional technique explained above focuses on the compression speed and the compression depth in chest compression training, and it is difficult to objectively evaluate whether or not the body movement of a trainee during training is appropriate. For this reason, in the conventional technique, although the training is intended to perform chest compressions at an appropriate compression speed and an appropriate compression depth, it is impossible to let the trainee know whether or not the trainee's body movement is appropriate during training.

According to one aspect, it is an object of the present invention to inform the trainee as to how the trainee is to move in order to optimize the compression speed and the compression depth during chest compressions.

### Solution to Problem

According to one aspect of the present disclosure, provided is a non-transitory computer-readable recording medium recorded with a cardiopulmonary resuscitation training program executable by a processor of an information processing apparatus, the cardiopulmonary resuscitation training program causing the processor to perform operations including evaluating a posture of a person who is performing chest compressions, based on posture information indicating the posture obtained from a posture detection apparatus and ideal posture information indicating an ideal posture for the chest compressions stored in a storage unit, to yield an evaluation result, and displaying the evaluation result on a display apparatus.

### Advantageous Effects of Invention

The trainee can understand how the trainee is to move in order to optimize the compression speed and the compression depth during chest compressions.

### BRIEF DESCRIPTION OF THE DRAWINGS

FIG. 1 is a drawing for explaining an overview of a cardiopulmonary resuscitation training system;

FIG. 2 is a drawing illustrating an example of a system configuration of the cardiopulmonary resuscitation training system;

FIG. 3 is a drawing illustrating an example of a hardware configuration of a posture detection apparatus;

FIG. 4 is a drawing illustrating an example of a hardware configuration of an information processing apparatus;

FIG. 5 is a drawing for explaining the functions of each apparatus provided in the cardiopulmonary resuscitation training system;

FIG. 6 is a flowchart for explaining processing performed by the information processing apparatus;

FIG. 7 is a drawing illustrating an example of a start screen of training of cardiopulmonary resuscitation;

FIG. 8 is a drawing illustrating an example of a training preparation screen of cardiopulmonary resuscitation;

FIG. 9 is an example of a screen displayed during training;

FIG. 10 is an example of an evaluation result screen;

FIG. 11 is an example of a screen displayed during training; and

FIG. 12 is an example of an evaluation result screen.

### DETAILED DESCRIPTION OF THE PREFERRED EMBODIMENTS

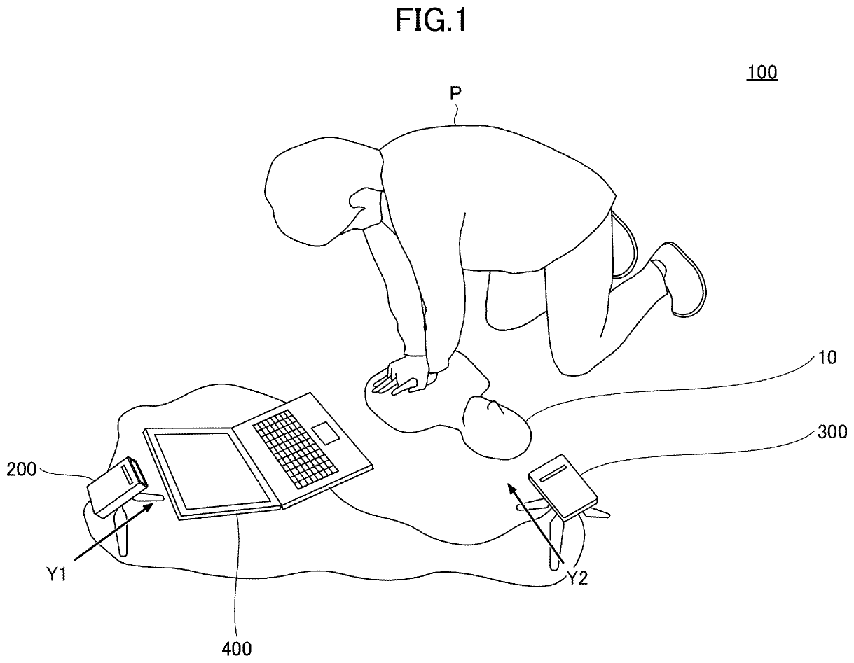

Hereinafter, the present embodiment is described with reference to the drawings. FIG. 1 is a drawing for explaining an overview of a cardiopulmonary resuscitation training system 100.

The cardiopulmonary resuscitation training system 100 according to the present embodiment includes posture detection apparatuses 200, 300 and an information processing apparatus 400.

In the cardiopulmonary resuscitation training system 100, a trainee P performs chest compressions on a manikin 10 according to the guidance provided by the information processing apparatus 400. The posture detection apparatus 200 and the posture detection apparatus 300 each detect the posture of the trainee P during chest compressions.

The posture detection apparatus 200 is placed to face the trainee P so as to face an arrow Y1 direction, and is configured to detect the posture of the trainee P when the trainee P is viewed from the front. The posture detection apparatus 300 is placed on a lateral side of the trainee P so as to face an arrow Y2 direction, and is configured to detect the posture of the trainee P when the trainee P is viewed from the side. The posture detection apparatus 200 and the posture detection apparatus 300 output information indicating the detected posture of the trainee P to the information processing apparatus 400. In the following explanation, the information indicating the posture of the trainee P detected by the posture detection apparatuses 200, 300 may be referred to as “posture information”.

The information processing apparatus 400 displays, on a display unit, the posture of the trainee P when the trainee P is seen from the front and the posture of the trainee P when the trainee P is seen from the side, on the basis of information that is output from the posture detection apparatus 200 and the posture detection apparatus 300.

Also, the information processing apparatus 400 compares the posture of the trainee P and the ideal posture by referring to information indicating the ideal posture during chest compressions (i.e., ideal posture information). Then, the information processing apparatus 400 displays a comparison result on the display unit and the like of the information processing apparatus 400.

In the present embodiment, in the manner as described above, a result obtained by comparing the ideal posture during chest compressions and the posture of the trainee P during training of the trainee P is displayed, so that the trainee P can objectively recognize the difference between the ideal posture and the actual posture of the trainee P.

According to the present embodiment, the movement of the trainee P can be evaluated objectively. For example, the movement of the trainee P is not subjectively evaluated by instructors.

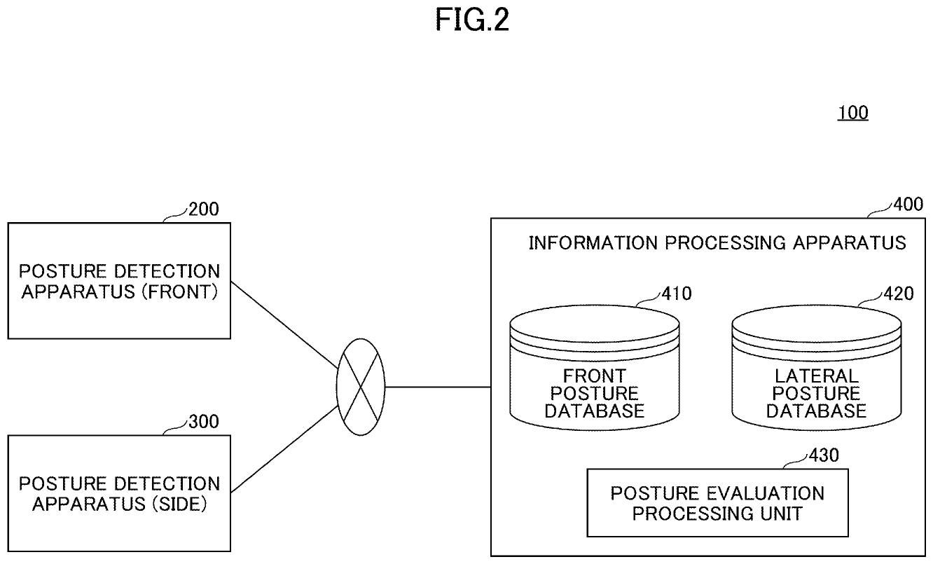

Subsequently, the system configuration of the cardiopulmonary resuscitation training system 100 according to the present embodiment is explained with reference to FIG. 2. FIG. 2 is a drawing illustrating an example of a system configuration of the cardiopulmonary resuscitation training system 100.

The cardiopulmonary resuscitation training system 100 according to the present embodiment includes the posture detection apparatus 200, the posture detection apparatus 300, and the information processing apparatus 400. The posture detection apparatus 200 and the information processing apparatus 400 are connected via a network, and the posture detection apparatus 300 and the information processing apparatus 400 are also connected via the network.

The information processing apparatus 400 according to the present embodiment includes a front posture database 410, a lateral posture database 420, and a posture evaluation processing unit 430.

The front posture database 410 stores front posture information indicating the ideal posture of chest compressions when a person who is performing the cardiopulmonary resuscitation is seen from the front. The lateral posture database 420 stores lateral posture information indicating the ideal posture of chest compressions when a person who is performing the cardiopulmonary resuscitation is seen from the side.

Hereinafter, the ideal posture of the present embodiment is explained.

In the cardiopulmonary resuscitation, the target ranges of the depth of compressions and the number of compressions are indicated in Resuscitation Guidelines of the Japan Resuscitation Council (JRC). In the cardiopulmonary resuscitation, the target range of the depth of chest compressions is 5 to 6 cm, and the target range of the number of compressions is about 100 to 120 times per minute.

Also, during chest compressions, it is preferable for a person giving chest compressions to extend the arms straight so that the arms of the person are perpendicular to the floor, look slightly forward instead of below, and put both hands, one on top of each other, to place the body weight on the heel of the palm. By maintaining such a posture, it is possible to reduce fatigue for keeping the depth of compressions and the number of compressions within the target range.

Therefore, in the present embodiment, the posture detection apparatus 200 and the posture detection apparatus 300 detect the postures of chest compressions performed by skilled experts of cardiopulmonary resuscitation such as healthcare professionals. Accordingly, in the present embodiment, the detection result of the posture detection apparatus 200 is defined as front posture information indicating the ideal posture as seen from the front, and the detection result of the posture detection apparatus 300 is defined as lateral posture information indicating the ideal posture as seen from the side.

In other words, the front posture information and the lateral posture information indicating the ideal posture of the present embodiment may be information obtained by tracking movement of the skeleton during a time in which the skilled expert performs chest compressions. In the present embodiment, for example, the time in which the chest compressions are performed is one minute. Therefore, the posture information indicating the ideal posture of the present embodiment may be referred to as moving picture data indicating movement of the skeleton of the skilled expert for the duration of one minute.

Also, the front posture information and the lateral posture information indicating the ideal posture of the present embodiment may be the skeleton detected from still pictures obtained while the skilled expert is performing chest compressions. The posture information indicating the ideal posture of the present embodiment may also be referred to as still picture data indicating the skeleton of the skilled expert.

Specifically, in the present embodiment, the front posture information is information including ideal ranges of flexion angle of left and right humeroradial joints during chest compressions and ideal ranges of rotation angles of left and right acromioclavicular joints with reference to the horizontal direction. Also, in the present embodiment, the lateral posture information is information including ideal angles of inclination angles of left and right upper limbs with reference to the ground and ideal angles of inclination angles of left and right lower limbs with reference to the ground.

In the present embodiment, the posture information indicating the ideal posture detected by the posture detection apparatus 200 is stored as the front posture information in the front posture database 410. Also, in the present embodiment, the posture information indicating the ideal posture detected by the posture detection apparatus 300 is stored as the lateral posture information in the lateral posture database 420.

The front posture database 410 and the lateral posture database 420 according to the present embodiment are both provided in the information processing apparatus 400, but the embodiment is not limited thereto. All or a part of the front posture database 410 and the lateral posture database 420 may be provided outside of the information processing apparatus 400.

The posture evaluation processing unit 430 according to the present embodiment compares the front posture of the trainee P detected by the posture detection apparatus 200 with the ideal posture indicated by the front posture information stored in the front posture database 410. Also, the posture evaluation processing unit 430 compares the lateral posture of the trainee P detected by the ideal posture detection apparatus 300 with the posture indicated by the lateral posture information stored in the lateral posture database 420.

Then, the information processing apparatus 400 displays the comparison result of the front posture and the comparison result of the lateral posture on the display unit. At this time, the information processing apparatus 400 may perform the comparison of the front posture and the comparison of the lateral posture at different and independent timings, or at the same time. Also, the information processing apparatus 400 may display the comparison result of the front posture and the comparison result of the lateral posture on the display unit at the same time, or at different and independent timings.

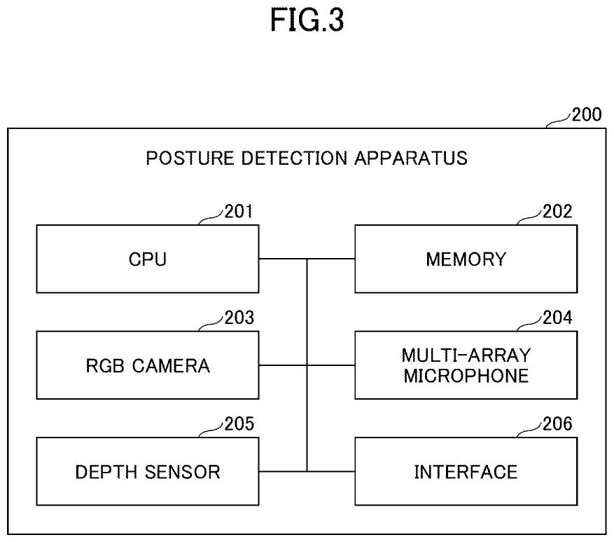

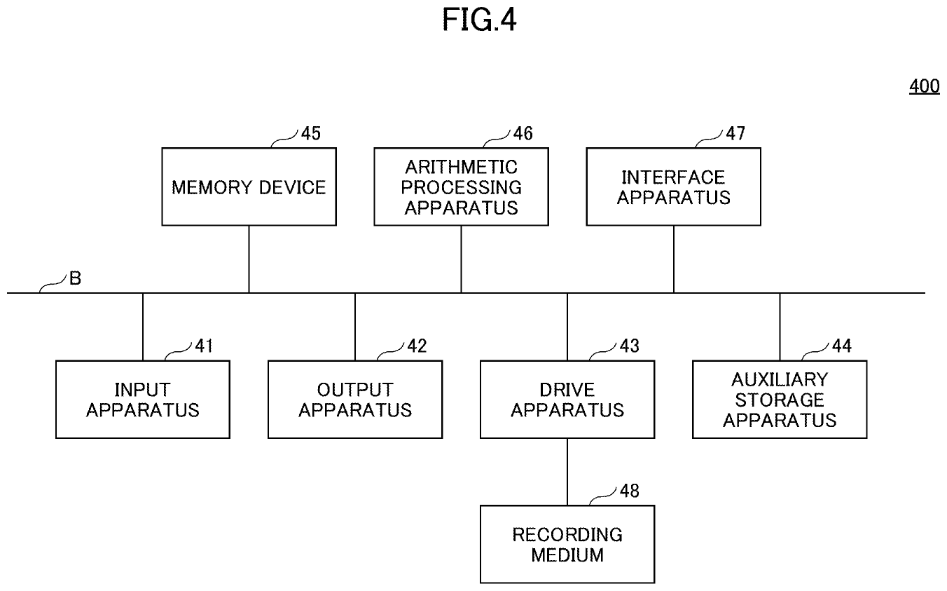

Subsequently, the hardware configuration of the posture detection apparatuses 200, 300 and the information processing apparatus 400 according to the present embodiment is explained with reference to FIG. 3 and FIG. 4.

FIG. 3 is a drawing illustrating an example of a hardware configuration of the posture detection apparatus. In the present embodiment, the hardware configuration of the posture detection apparatus 200 and the hardware configuration of the posture detection apparatus 300 are the same, and hereinafter, the hardware configuration of the posture detection apparatus 200 is explained as an example.

The posture detection apparatus 200 according to the present embodiment includes a central processing unit (CPU) 201, a memory 202, a red-green-blue (RGB) camera 203, a multi-array microphone 204, a depth sensor 205, and an interface 206, which are connected via a bus with each other.

The CPU 201 controls the overall operation of the posture detection apparatus 200. The memory 202 stores data used in the operation of the CPU 201 and data obtained by the operation. The RGB camera 203 captures an RGB image and adopts the RGB image as image data. The multi-array microphone 204 include multiple omnidirectional microphones arranged on a plane.

The depth sensor 205 detects an object as a three-dimensional object by irradiating the object with laser light such as infrared rays while changing the irradiation position of the laser light.

The interface 206 is an interface for the posture detection apparatus 200 to communicate with another apparatus. Specifically, the another apparatus is the information processing apparatus 400.

Subsequently, the hardware configuration of the information processing apparatus 400 according to the present embodiment is explained with reference to FIG. 4. FIG. 4 is a drawing illustrating an example of the hardware configuration of an information processing apparatus 400. The information processing apparatus 400 according to the present embodiment is a computer including an input apparatus 41, an output apparatus 42, a drive apparatus 43, an auxiliary storage apparatus 44, a memory device 45, an arithmetic processing apparatus 46, and an interface apparatus 47, which are connected with each other via a bus.

The input apparatus 41 is an apparatus for inputting various kinds of information, and implemented with, for example, a keyboard, a touch panel, and the like. The output apparatus 42 is an apparatus for outputting various kinds of information, and implemented with, for example, a display unit, and the like. The interface apparatus 47 is used to connect to the network.

A posture evaluation program for implementing the posture evaluation processing unit 430 is at least a part of various programs for controlling the information processing apparatus 400. For example, the posture evaluation program is provided in a form of a storage medium 48 or is downloaded from the network. The storage medium 48 recorded with the posture evaluation program may be various types of storage media such as a storage medium for optically, electrically, or magnetically recording information, and a semiconductor memory for electrically recording information such as a read-only memory (ROM), a flash memory, or the like.

When the storage medium 48 recorded with the posture evaluation program is set on the drive apparatus 43, the posture evaluation program is installed from the storage medium 48 via the drive apparatus 43 to the auxiliary storage apparatus 44. The posture evaluation program downloaded from the network is installed via the interface apparatus 47 to the auxiliary storage apparatus 44.

The auxiliary storage apparatus 44 implementing the front posture database 410 and the lateral posture database 420 stores the posture evaluation program installed to the information processing apparatus 400, and stores various kinds of files, data, and the like used by the information processing apparatus 400. The memory device 45 reads the posture evaluation program from the auxiliary storage apparatus 44 and stores the posture evaluation program when the information processing apparatus 400 starts. Then, the arithmetic processing apparatus 46 implements various kinds of processing, as explained later, according to a posture evaluation display program stored in the memory device 45.

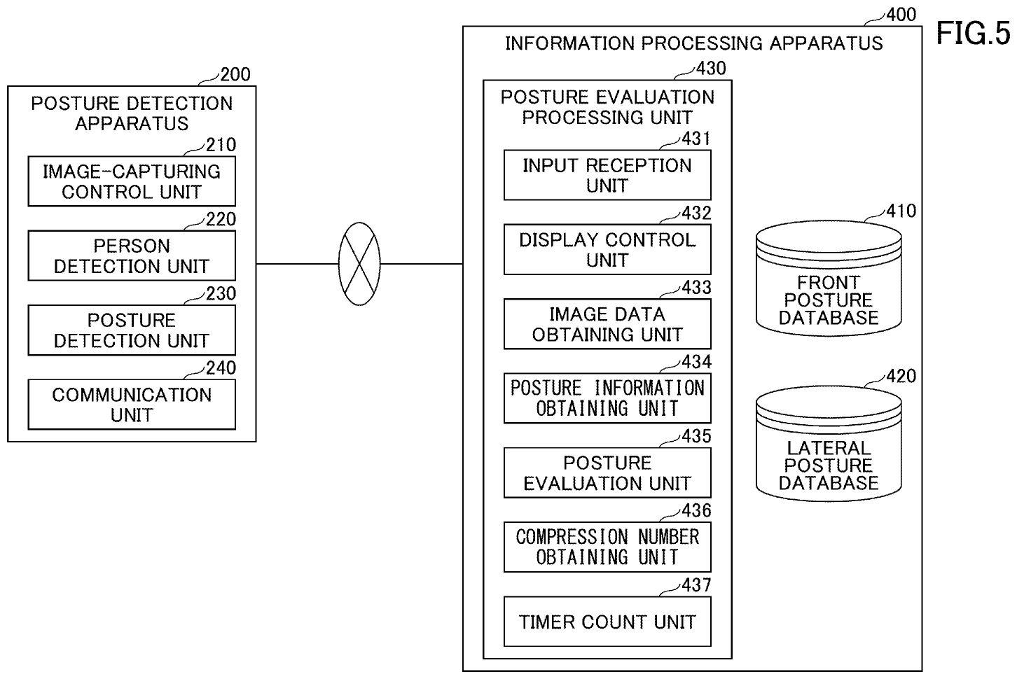

Subsequently, the function of each apparatus in the cardiopulmonary resuscitation training system 100 according to the present embodiment are explained with reference to FIG. 5. FIG. 5 is a drawing for explaining the functions of each apparatus in the cardiopulmonary resuscitation training system.

First, the functions of the posture detection apparatuses 200, 300 are explained. Since the posture detection apparatus 200 and the posture detection apparatus 300 according to the present embodiment have functions similar to each other, the functions of the posture detection apparatus 200 are explained as one example with reference to FIG. 5

The posture detection apparatus 200 according to the present embodiment includes an image-capturing control unit 210, a person detection unit 220, a posture detection unit 230, and a communication unit 240. The functions of the above units explained above can be achieved by causing the CPU 201 to read and execute programs stored in the memory 202.

The image-capturing control unit 210 controls the RGB camera 203 to capture images and use the images as image data. The person detection unit 220 detects whether or not an object detected by the depth sensor 205 is a human body.

When the detected object is a human body, the posture detection unit 230 detects the posture of the human body and outputs posture information. The posture information according to the present embodiment is information indicating movements of predetermined positions of the skeleton of the human body obtained as a group of three-dimensional coordinates indicating the predetermined positions of the skeleton of the human body. In the present embodiment, for example, information indicating the movements at 24 positions of the skeleton of the human body may be obtained as the posture information.

The communication unit 240 transmits the posture information indicating the posture detected by the posture detection unit 230 to the information processing apparatus 400.

Subsequently, the functions of the information processing apparatus 400 are explained. The information processing apparatus 400 according to the present embodiment includes a posture evaluation processing unit 430. The posture evaluation processing unit 430 is implemented by causing the arithmetic processing apparatus 46 to execute the posture evaluation program installed to the information processing apparatus 400.

The posture evaluation processing unit 430 includes an input reception unit 431, a display control unit 432, an image data obtaining unit 433, a posture information obtaining unit 434, a posture evaluation unit 435, a compression number obtaining unit 436, and a timer count unit 437.

The input reception unit 431 receives various kinds of input to the information processing apparatus 400. Specifically, for example, the input reception unit 431 receives an input of an operation for instructing the start of training of cardiopulmonary resuscitation.

The display control unit 432 controls display of the display unit (i.e., a display apparatus). Specifically, for example, the display control unit 432 displays a screen indicating the posture of the trainee P in training, a screen indicating an evaluation result of the posture of the trainee P, and the like on the display apparatus.

The image data obtaining unit 433 obtains image data from the posture detection apparatuses 200, 300. The image data obtained here is RGB image data.

The posture information obtaining unit 434 obtains the posture information transmitted from the posture detection apparatuses 200, 300. Specifically, the posture information obtaining unit 434 obtains, from the posture detection apparatus 200, the posture information of the posture of the trainee P when the trainee P is seen from the front, and obtains, from the posture detection apparatus 300, the posture information of the posture of the trainee P when the trainee P is seen from the side.

The posture evaluation unit 435 compares the posture information obtained by the posture information obtaining unit 434 with information indicating the ideal posture stored in the front posture database 410 and the lateral posture database 420.

Specifically, the posture evaluation unit 435 determines whether the flexion angle of left and right humeroradial joints of the trainee P indicated in the posture information obtained from the posture detection apparatus 200 by the posture information obtaining unit 434 are within the ranges of the ideal angles indicated in the front posture information. Also, the posture evaluation unit 435 determines whether the rotation angles of left and right acromioclavicular joints of the trainee P indicated in the posture information obtained from the posture detection apparatus 300 with reference to the horizontal direction indicated in the posture information are within the ranges of the ideal angles indicated in the front posture information.

The posture evaluation unit 435 determines whether inclination angles of left and right upper limbs of the trainee P with reference to the ground indicated in the posture information are within the ranges of the ideal angles indicated by the lateral posture information.

The compression number obtaining unit 436 obtains the number of times the trainee P compressed the manikin 10 with the depth of compressions being within the target range during training.

Hereinafter, the manikin 10 is explained. The manikin 10 according to the present embodiment has a mechanism that makes a clicking sound when the portion corresponding to the sternum is pressed to a depth that is within the target range.

The compression number obtaining unit 436 obtains a compression number by counting, as the number of times the trainee P has appropriately performed compressions, the number of times the clicking sounds are detected. The detection of clicking sounds may be performed by a sound collecting apparatus provided in the information processing apparatus 400, or may be performed by the multi-array microphone 204 included in the posture detection apparatus 200.

The timer count unit 437 includes a timer with a training time being set, and when the timer count unit 437 receives a start instruction of training, the timer count unit 437 starts counting by decreasing the value of the timer with regular intervals. The timer count unit 437 may be implemented by a clock function of the information processing apparatus 400.

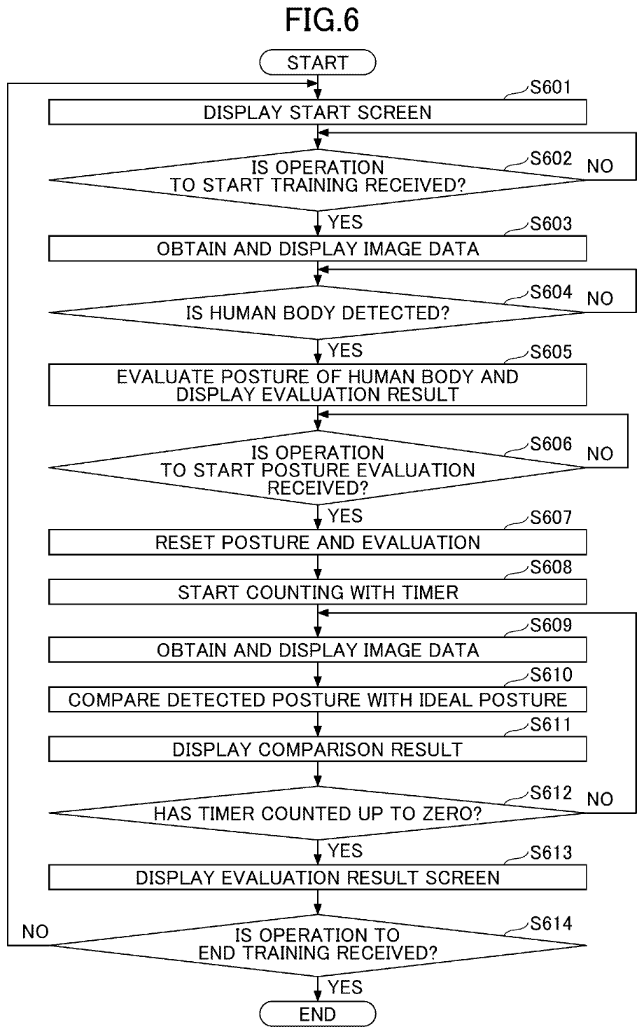

Subsequently, processing performed by the information processing apparatus 400 according to the present embodiment is explained with reference to FIG. 6. FIG. 6 is a flowchart for explaining processing of the information processing apparatus 400. FIG. 6 illustrates an example of processing for evaluating the posture of the trainee P when the trainee P is seen from the front by using the posture information obtained from the posture detection apparatus 200.

When the posture evaluation program is started, the information processing apparatus 400 according to the present embodiment displays the start screen of training of cardiopulmonary resuscitation according to the display control unit 432 of the posture evaluation processing unit 430 (step S601).

Subsequently, the posture evaluation processing unit 430 determines whether the input reception unit 431 receives an operation to start training (step S602). In a case where the input reception unit 431 does not receive the operation in step S602, the posture evaluation processing unit 430 waits until the input reception unit 431 receives the operation.

In a case where the input reception unit 431 receives the operation in step S602, the posture evaluation processing unit 430 causes the image data obtaining unit 433 to receive RGB image data from the posture detection apparatus 200, and causes the display control unit 432 to display the RGB image data on the display unit (step S603).

Subsequently, the posture evaluation processing unit 430 determines whether the posture detection apparatus 200 detects a human body (step S604). In the present embodiment, when the posture detection apparatus 200 detects a human body, the posture detection apparatus 200 may output a notification indicating detection of a human body to the information processing apparatus 400. When the input reception unit 431 receives the notification, the posture evaluation processing unit 430 may determine that a human body has been detected.

In step S604, in a case where a human body is not detected, the posture evaluation processing unit 430 waits.

When a human body is detected in step S604, the posture evaluation processing unit 430 causes the posture information obtaining unit 434 to obtain the posture information from the posture detection apparatus 200, causes the posture evaluation unit 435 to compare the obtained posture information with the front posture information (i.e., the ideal posture), and causes the display control unit 432 to display, on the display unit, a training preparation screen including the comparison result and the evaluation result of the posture (step S605). In this case, an operation button for instructing the start of posture evaluation of chest compressions may be displayed on the training preparation screen.

Subsequently, the information processing apparatus 400 determines whether the input reception unit 431 of the posture evaluation processing unit 430 has received an operation for instructing the start of posture evaluation of chest compressions (step S606). In a case where the input reception unit 431 does not receive the operation in step S606, the posture evaluation processing unit 430 waits.

In a case where the input reception unit 431 receives the operation in step S606, the posture evaluation unit 435 of the posture evaluation processing unit 430 resets an evaluation result (step S607). Specifically, information displayed as an evaluation result is erased.

Subsequently, the posture evaluation processing unit 430 causes the timer count unit 437 to start counting with the timer (step S608).

Subsequently, the posture evaluation processing unit 430 causes the image data obtaining unit 433 to obtain RGB image data from the posture detection apparatus 200, and causes the display control unit 432 to display the obtained RGB image data on the display unit (step S609).

Subsequently, the posture evaluation processing unit 430 causes the posture information obtaining unit 434 to obtain the posture information, and causes the posture evaluation unit 435 to compare the obtained posture information of the trainee P with the front posture information (i.e., the ideal posture) (step S610). Subsequently, the posture evaluation processing unit 430 causes the display control unit 432 to display, on the display unit, an evaluation result screen indicating the comparison result and the evaluation result of the posture (step S611).

Subsequently, the posture evaluation processing unit 430 causes the timer count unit 437 to determine whether the timer has counted up to zero (step S612). In a case where the timer count unit 437 determines that the timer has not counted up to zero in step S612, the posture evaluation processing unit 430 returns back to step S609.

In a case where the timer count unit 437 determines that the timer has counted up to zero in step S612, the posture evaluation processing unit 430 causes the posture evaluation unit 435 to summarize the evaluation results of the training, and causes the display control unit 432 to display, on the display unit, the evaluation result of the entire training (step S613).

Subsequently, the information processing apparatus 400 determines whether the input reception unit 431 receives an operation to end the training (step S614). In a case where the input reception unit 431 does not receive the operation in step S614, the posture evaluation processing unit 430 returns back to step S601.

In a case where the input reception unit 431 receives the operation in step S614, the posture evaluation processing unit 430 ends the processing.

The processing for evaluating the posture information detected by the posture detection apparatus 300 is similar to the processing of FIG. 6 except that lateral posture information stored in the lateral posture database 420 is referred to.

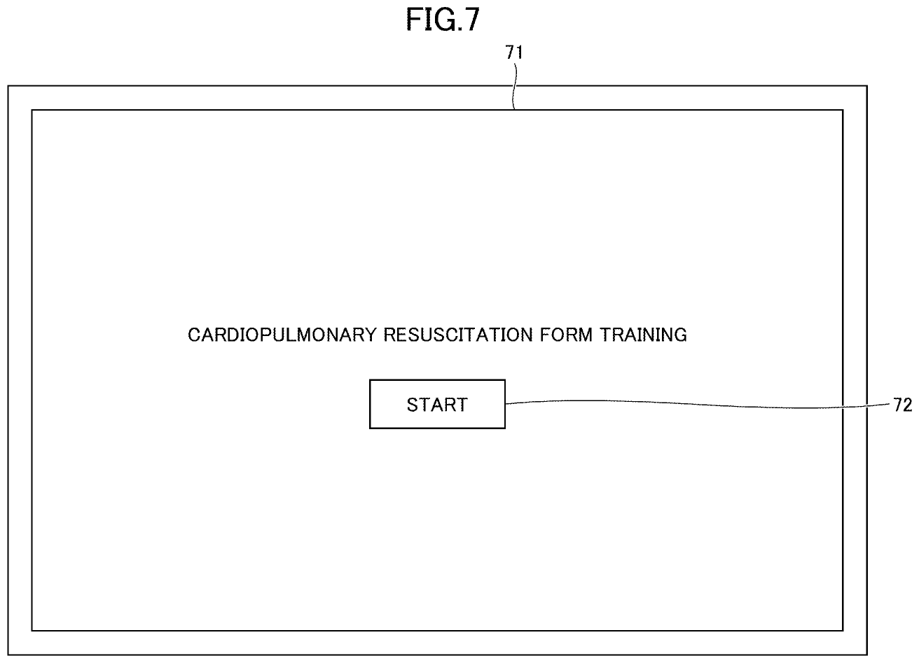

Subsequently, a transition of a screen displayed on the display unit in the processing of FIG. 6 is explained with reference to FIG. 7 to FIG. 10. FIG. 7 is a drawing illustrating an example of a start screen of training of cardiopulmonary resuscitation. A screen 71 illustrated in FIG. 7 is an example of a start screen of training displayed on the display unit in step S601 of FIG. 6.

The operation button 72 for instructing a start of cardiopulmonary resuscitation is displayed on the screen 71, and in a case where an operation for selecting the operation button 72 is performed, the posture evaluation processing unit 430 starts the processing illustrated in FIG. 6.

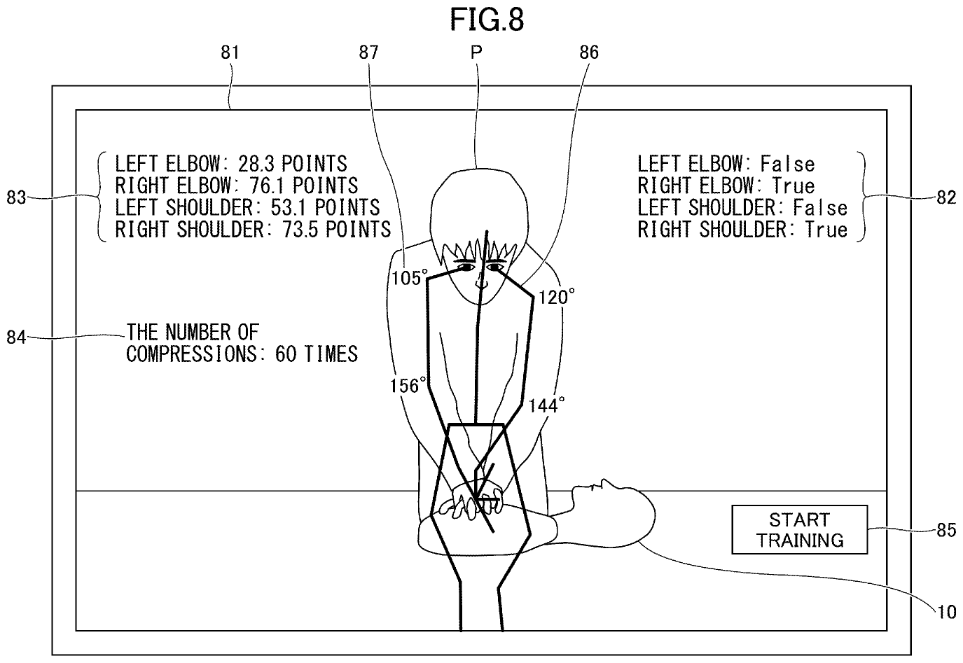

FIG. 8 is a drawing illustrating an example of a screen 81 (i.e., a training preparation screen) of cardiopulmonary resuscitation. The screen 81 illustrated in FIG. 8 is an example of a training preparation screen displayed in step S606 of FIG. 6.

The screen 81 includes an image of the trainee P, display areas 82, 83, and 84, the operation button 85, an image 86 indicating the skeleton of the trainee P, and numerical values 87.

A result obtained by comparing the flexion angle of humeroradial joints of the trainee P and the ideal ranges and determining whether the flexion angles are within the ideal ranges is displayed in the display area 82. A result obtained by comparing the rotation angles of left and right acromioclavicular joints of the trainee P and the ideal ranges and determining whether the rotation angles of left and right acromioclavicular joints are within the ideal ranges is displayed in the display area 82.

In the present embodiment, as a result of comparing the flexion angle of left and right humeroradial joints of the trainee P with the ideal ranges, in a case where the flexion angles are within the ideal ranges, “True” is displayed in the display area 82, and in a case where the flexion angles are out of the ideal ranges, “False” is displayed in the display area 82. In the present embodiment, as a result of comparing the rotation angles of left and right acromioclavicular joints of the trainee P with the ideal ranges, in a case where the rotation angles are within the ideal ranges, “True” is displayed in the display area 82, and in a case where the rotation angles are out of the ideal ranges, “False” is displayed in the display area 82.

Information obtained by quantifying a result obtained by comparing the flexion angle of left and right humeroradial joints of the posture at the start of the training of the trainee P with the ideal ranges and information obtained by quantifying a result obtained by comparing the rotation angles of left and right acromioclavicular joints of the posture at the start of the training of the trainee P with the ideal ranges are displayed in the display area 83.

Specifically, the numerical value displayed in the display area 83 may be numerical values obtained by quantifying a difference between the angle derived from the posture of the trainee P and the most preferable value in the ideal range.

A target compression number is displayed in the display area 84. The operation button 85 is an operation button for instructing the start of posture evaluation. In other words, the operation button 85 is the operation button for instructing the start of counting with the timer.

The image 86 is an image indicating the skeleton of the trainee P. The numerical value 87 indicates the flexion angle of left and right humeroradial joints of the trainee P and the rotation angles of left and right acromioclavicular joints of the trainee P, which are derived from the image 86.

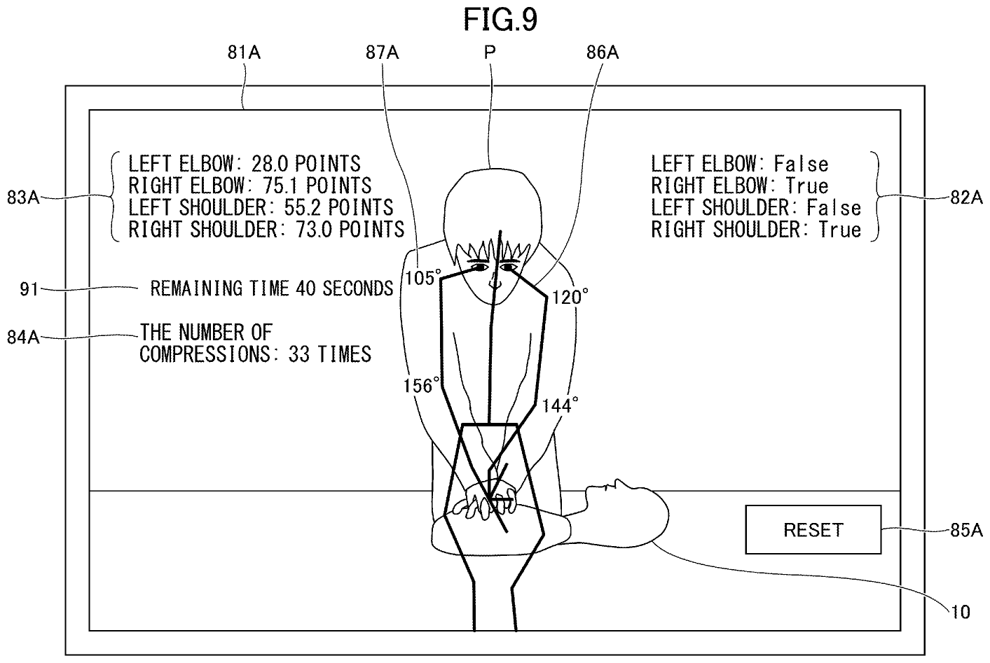

FIG. 9 is an example of a screen 81A displayed during training. The screen 81A illustrated in FIG. 9 is an example of a screen displayed after the operation button 85 is operated in the screen 81 illustrated in FIG. 8 to start counting with the timer.

The screen 81A includes an image of the trainee P, display areas 82A, 83A, and 84A, an operation button 85A, an image 86A indicating the skeleton of the trainee P, numerical values 87A, and a display area 91.

A result obtained by comparing the flexion angle of humeroradial joints of the trainee P with the ideal ranges and a result obtained by comparing the rotation angles of left and right acromioclavicular joints with the ideal ranges are displayed in the display area 82A. In the example of FIG. 9, it can be understood that, when the remaining time of the training time becomes 40 seconds, the flexion angle of the left humeroradial joint and the rotation angle of the left acromioclavicular joint are out of the ideal ranges, and the flexion angle of the right humeroradial joint and the rotation angle of the right acromioclavicular joint are within the ideal ranges.

Information obtained by quantifying a result obtained by comparing the flexion angle of left and right humeroradial joints of the trainee P during training of the trainee P with the ideal ranges and information obtained by quantifying a result obtained by comparing the rotation angles of left and right acromioclavicular joints of the trainee P during training of the trainee P with the ideal ranges are displayed in the display area 83A.

In other words, the numerical values displayed in the display area 83A are index values indicating the degree of closeness of the posture of the trainee P during training of the trainee P to the ideal posture. In the present embodiment, as the index value becomes closer to 100, the posture of the trainee P during training of the trainee P becomes closer to the ideal posture.

Specifically, the index values displayed in the display area 83A may be calculated, for example, as ratios of a time in which the flexion angle of left and right humeroradial joints and the rotation angles of left and right acromioclavicular joints are within the ideal ranges to a time counted by the timer.

In the example of FIG. 9, when the remaining time of the training time is 40 seconds, the index values indicating the degree of closeness of the posture of the trainee P to the ideal posture are 28.0 for the left elbow, 75.1 for the right elbow, 55.2 for the left shoulder, and 73.0 for the right shoulder. Therefore, it can be understood that, when the remaining time of the training time is 40 seconds, the flexion angle of the right humeroradial joint and the rotation angle of the right acromioclavicular joint of the trainee P are close to the ideal angles, and the flexion angle of the left humeroradial joint and the rotation angle of the left acromioclavicular joint of the trainee P are farther from the ideal angles as compared with the right side.

The number of times the trainee P has performed compressions to the manikin 10 since the counting with the timer has started is indicated in the display area 84A. In other words, the number of times the clicking sounds are detected from the manikin 10 is indicated in the display area 84A.

The operation button 85A is an operation button for resetting the processing until then. In other words, the operation button 85A is an operation button for interrupting the training.

The display area 91 indicates the remaining time of the training time that is set in the timer. In the example of FIG. 9, it can be understood that the remaining time is seconds.

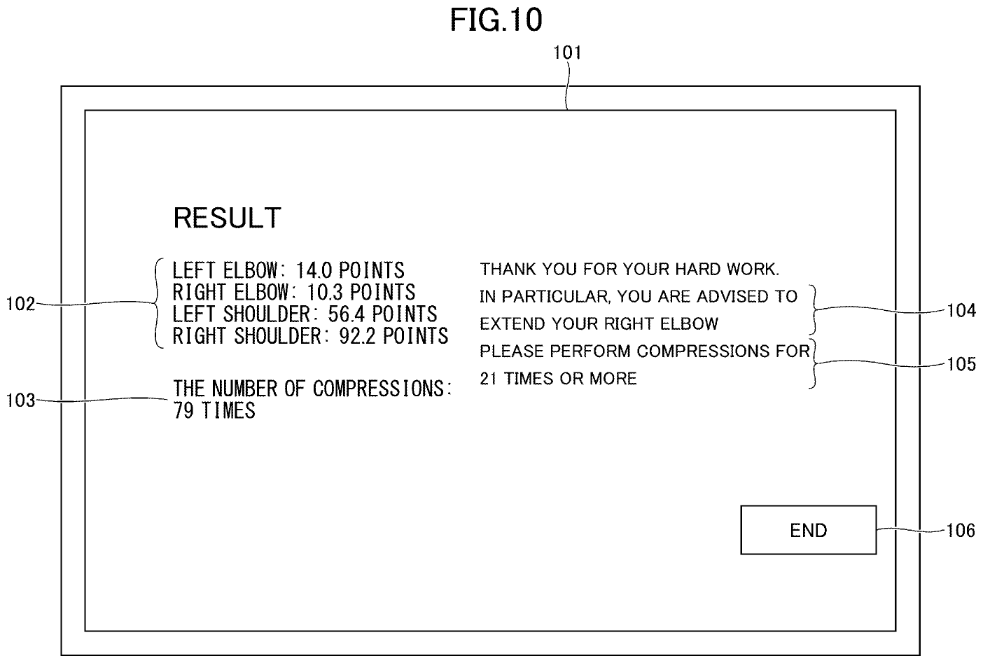

FIG. 10 is an example of a screen 101 (i.e., an evaluation result screen). For example, the screen 101 as illustrated in FIG. 10 is an example of an evaluation result screen displayed in step S611 of FIG. 6.

The screen 101 includes display areas 102, 103, 104, and 105, and an operation button 106. The display area 102 indicates the evaluation result of the posture during training. Specifically, the numerical values displayed in the display area 102 are index values indicating the degree of closeness, to the ideal posture, of the posture during training when the trainee P is seen from the side.

The number of times the trainee P has performed compressions to the manikin 10 since the counting with the timer has started is indicated in the display area 103. In other words, the number of times the clicking sounds are detected from the manikin 10 is indicated in the display area 103.

The display area 104 displays advice information about the posture. Specifically, a warning message may be displayed in the display area 104 with regard to joints of which the numerical values displayed in the display area 102 are less than a predicted value.

The advice information about the compression number is displayed in the display area 105. Specifically, in a case where the compression number does not attain a target value, a message including the number of times in shortage may be displayed in the display area 105. The operation button 106 is an operation button for ending the training of cardiopulmonary resuscitation.

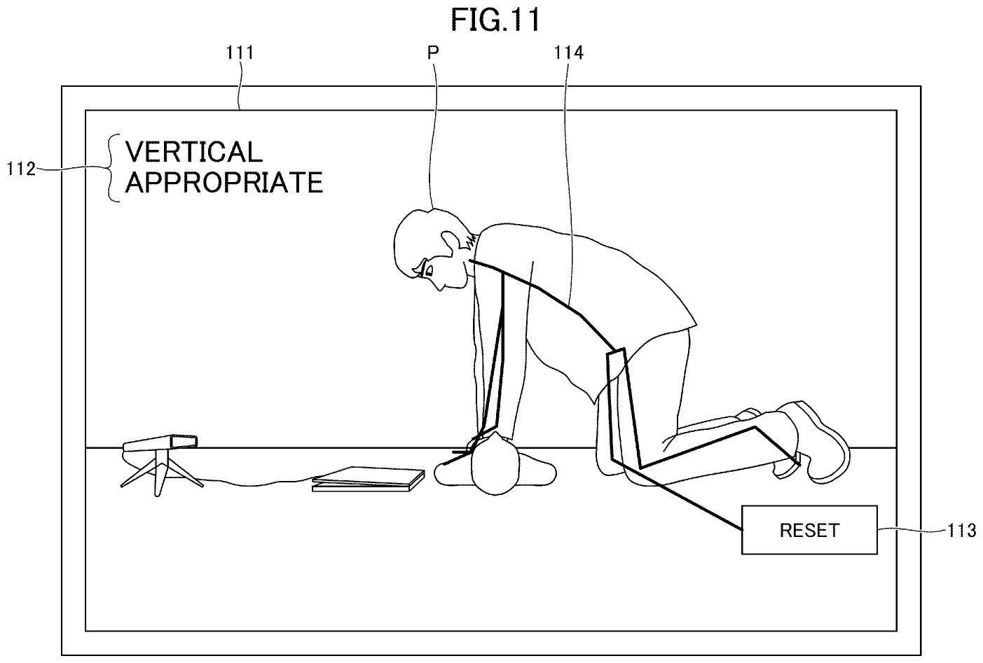

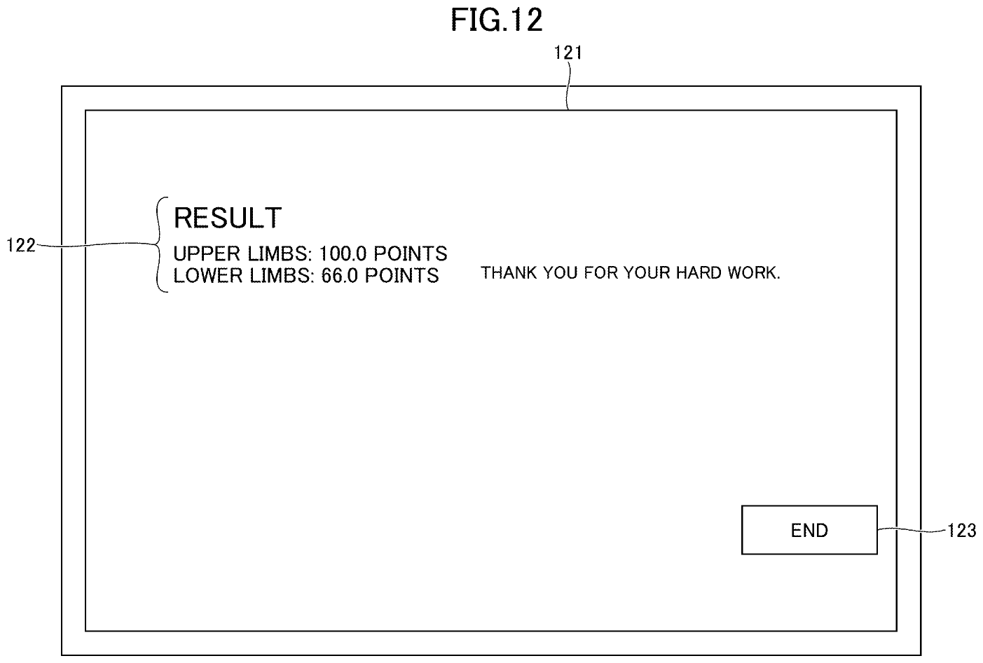

Subsequently, an example of display in a case where the posture information detected by the posture detection apparatus 300 and the lateral posture information are compared is explained with reference to FIG. 11 and FIG. 12.

FIG. 11 is an example of a screen 111 displayed during training. The screen 111 illustrated in FIG. 11 is an example of a screen displayed after the counting performed with the timer is started.

The screen 81A includes an image of the trainee P when the trainee P is seen from the side, a display area 112, an operation button 113, and an image 114 indicating the skeleton of the trainee P.

A result obtained by determining whether the inclination angles of the upper limbs and the lower limbs with reference to a direction perpendicular to the ground are within the ideal ranges is displayed in the display area 112. In the example of FIG. 11, it can be understood that the inclination angles of the upper limbs and the lower limbs with reference to the ground are within the ideal ranges. The operation button 113 is an operation button for resetting the processing until the current time. In other words, the operation button 113 is an operation button for interrupting the training.

FIG. 12 is an example of a screen 121 (i.e., an evaluation result screen). The screen 121 illustrated in FIG. 12 includes a display area 122 and an operation button 123. The operation button 123 is an operation button for instructing the end of the training.

A posture evaluation result of the trainee P when the trainee P is seen from the side is illustrated in the display area 122. Specifically, the numerical values displayed in the display area 122 are obtained by quantifying a result obtained by comparing the inclination angles of the upper limbs and the lower limbs of the trainee P during training with the ideal ranges. In other words, the numerical values displayed in the display area 122 are index values indicating the degree of closeness, to the ideal posture, of the posture of the trainee P when the trainee P is seen from the side. In the present embodiment, as the index value becomes closer to 100, the posture of the trainee P during training of the trainee P becomes closer to the ideal posture.

Specifically, the index values displayed in the display area 122 may be calculated, for example, as ratios of a time in which the inclination angles of the upper limbs and the lower limbs are within the ideal ranges to a time counted by the timer.

In the example of FIG. 12, the index values indicating the degree of closeness of the posture of the trainee P during training to the ideal posture are 100 for the upper limb and 66 for the lower limb. Therefore, it is understood that the posture of the trainee P is ideal with regard to the upper limb of the trainee P, but the trainee P is expected to improve the posture of the lower limb.

In the manner as described above, according to the present embodiment, the movement of the body of the trainee P during chest compressions can be objectively evaluated. According to the present embodiment, the evaluation result of the movement of the body of the trainee P is notified to the trainee P, so that the trainee P can understand whether the movement of the body of the trainee P during the training is appropriate and how the trainee P should correct the movement of the body of the trainee P.

Therefore, according to the present embodiment, in the training of cardiopulmonary resuscitation, the posture of the trainee P is not subjectively evaluated by instructors.

Further, in the present embodiment, ordinary citizens can learn practical skills with accurate posture for the cardiopulmonary resuscitation that is performed on a person in cardiopulmonary arrest. Further, in the present embodiment, even without any instructor instructing practical skills, the trainee P learns the practical skills on the basis of the evaluation result of the posture, so that the trainee P can acquire accurate practical skills.

In the manner as described above, according to the present embodiment, accurate practical skills of cardiopulmonary resuscitation can be widely spread. Therefore, according to the present embodiment, for example, when an ordinary citizen sees a person in cardiopulmonary arrest, the citizens are more likely to make up their minds to “perform cardiopulmonary resuscitation”.

In the present embodiment, the evaluation of the posture of the trainee P when the trainee P is seen from the front and the evaluation of the posture of the trainee P when the trainee P is seen from the side are performed separately, but the embodiment is not limited thereto.

The evaluation of the front posture and the evaluation of the lateral posture may be performed in the same way in a single training session. In that case, the evaluation of the posture information obtained from the posture detection apparatus 200 and the evaluation of the posture information obtained from the posture detection apparatus 300 may be performed in parallel, and both of the evaluation results may be displayed on the same screen.

Also, the information processing apparatus 400 according to the present embodiment may include, for example, a metronome function for outputting sound at a constant tempo to notify, to the trainee, a rhythm for performing compressions to the manikin 10. With such a function, the trainee P can find the tempo of chest compressions.

Also, in the present embodiment, the screens illustrated in FIG. 7 to FIG. 12 are displayed on the display unit of the information processing apparatus 400, but the embodiment is not limited thereto. The screens illustrated in FIG. 7 to FIG. 12 may be displayed on a display apparatus capable of communicating with the information processing apparatus 400.

The present invention is not limited to the embodiment specifically disclosed above, and can be modified and changed in various manners without departing from the scope of the claimed subject matter.

What is claimed is:

1. A non-transitory computer-readable recording medium recorded with a cardiopulmonary resuscitation training program executable by a processor of an information processing apparatus, the cardiopulmonary resuscitation training program causing the processor to perform operations comprising:
evaluating a posture of a person who is performing chest compressions, based on posture information indicating the posture obtained from a posture detection apparatus and ideal posture information indicating an ideal posture for the chest compressions stored in a storage unit, to yield an evaluation result; and
displaying the evaluation result on a display apparatus.

2. The non-transitory computer-readable recording medium according to claim 1, wherein the posture information is information indicating the posture of the person when the person who is performing the chest compressions is seen from a front of the person.

3. The non-transitory computer-readable recording medium according to claim 1, wherein the ideal posture information includes an ideal range of a flexion angle of a predetermined joint for the chest compressions,
the posture information includes a flexion angle of the predetermined joint of the person who is performing the chest compressions, and
the evaluation result includes information indicating whether the flexion angle of the predetermined joint of the person is within the ideal range.

4. The non-transitory computer-readable recording medium according to claim 3, wherein the evaluation result includes an index value indicating a degree of closeness of the posture of the person to the ideal posture.

5. The non-transitory computer-readable recording medium according to claim 1, wherein the posture information is information indicating a posture of the person when the person who is performing the chest compressions is seen from a side of the person.

6. The non-transitory computer-readable recording medium according to claim 4, wherein the ideal posture information includes an ideal range of an inclination angle of at least one of an upper limb or a lower limb of the person with reference to a ground during the chest compressions,
the posture information includes an inclination angle of the at least one of the upper limb or the lower limb of the person with reference to the ground during the chest compressions, and
the evaluation result includes information indicating whether the inclination angle of the at least one of the upper limb or the lower limb of the person with reference to the ground is within the ideal range.

7. The non-transitory computer-readable recording medium according to claim 1, wherein the evaluation result and image data obtained by capturing an image of the person who is performing the chest compressions are displayed on the display apparatus.

8. A cardiopulmonary resuscitation training method for causing an information processing apparatus to perform operations comprising:
evaluating a posture of a person who is performing chest compressions, based on posture information indicating the posture obtained from a posture detection apparatus and ideal posture information indicating an ideal posture for the chest compressions stored in a storage unit, to yield an evaluation result; and
displaying the evaluation result on a display apparatus.

9. A cardiopulmonary resuscitation training apparatus comprising a processor configured to perform the cardiopulmonary resuscitation training method of claim 8.

10. A cardiopulmonary resuscitation training system comprising:
a posture detection apparatus; and
an information processing apparatus including a processor configured to perform the cardiopulmonary resuscitation training method of claim 8.
